(19) United States
(12) Patent Application Publication
(10) Pub. No.: US 2007/0197424 A1
(43) Pub. Date: Aug. 23, 2007

(54) Glatiramer acetate for use as an immuno-modulatory agent

(76) Inventors: Scott L. Friedman, Scarsdale, NY (US); Rifaat Safadi, Nazareth (IL)

Correspondence Address: FLEIT KAIN GIBBONS GUTMAN BONGINI & BIANCO, 21355 EAST DIXIE HIGHWAY, SUITE 115, MIAMI, FL, 33180, US

(21) Appl. No.: 10/571,803

(22) PCT Filed: Sep. 14, 2004
(86) PCT No.: PCT/IL04/00848

§ 371 Date: Jan. 8, 2007

(30) Foreign Application Priority Data
Sep. 16, 2003 (IL) 157953
Oct. 1, 2003 (IL) 158212

(51) Int. Cl. A61K 38/16

(52) U.S. Cl. 514/2

(57) ABSTRACT

A method for the treatment of hepatic fibrotic injuries caused by various diseases, viral infections or toxic agents which involves the use of glatiramer acetate as an immuno-modulatory agent. The diseases to be treated are hepatic fibrosis and hepatic cellular carcinomas. Also disclose is the use of glatiramer acetate in the treatment of inflammatory bowel diseases. Additionally, disclosed are methods for screening for immuno-modulatory agents which are useful in the treatment of hepatic fibrosis, hepatic cellular carcinomas and inflammatory bowel diseases.

### FIELD OF THE INVENTION

The present invention relates to use of immuno-modulatory agents, particularly glatiramer acetate (Copaxone [also known as Copolymer-1], Teva Ltd.), optionally in combination with other immune active agents such as IL-2, in the treatment of hepatic fibrosis.

### BACKGROUND OF THE INVENTION

The present inventors have previously reported a role of increased CD8 and decreased CD4 lymphocyte subsets in mediating hepatic fibrosis which is attenuated by IL-10 [Safadi, R., et al., Poster #387, AASLD, Boston 2002; Safadi, R., et al., Oral Presentation #610, AASLD, Boston 2002 and Safadi, R., et al., Gastroenterology, In Press 2004]. This issue was approached by generating a transgenic mouse secreting rat interleukin-10 (rIL-10) in hepatocytes to assess the impact of sustained local expression of the cytokine on hepatic fibrogenesis in two distinct animal models. Having identified an antifibrotic effect of rIL-10, the influence of this cytokine on lymphocyte subsets was characterized, and a specific reduction of CD8 lymphocytes was identified. Finally, the fibrogenic activity of CD8 lymphocytes isolated from animals with liver injury was tested by adoptive transfer into naïve animals. NK cells, however, are typically activated by CD4 and dendritic (CD11c+) antigen-presenting cells; in mouse they express both inhibitory killing immunoglobulin receptor (iKIR) and activation KIR (aKIR) specific for class Ia MHC molecules, but killing only occurs upon loss of class Ia MHC on target cells.

Thus, in the experimental models used by the inventors, which will be detailed hereafter, NK cells have anti-fibrotic activity via their activation combined with increased killing of activated HSC's. In vivo there may be opposing effects of CD4 loss, which favours fibrogenesis, and NK activation, which is anti-fibrogenic. These findings significantly broaden the understanding of immune mediation of fibrosis and point to manipulation of CD4, CD8 and NK subsets as potential options in modulating fibrosis therapeutically, and form the basis for the present invention.

Hepatic fibrosis is exhibited in the accumulation of connective tissue in the liver. Fibrosis is the result of chronic injury to the liver, regardless of etiology. During the hepatic injury response, hepatic stellate cells transdifferentiate, or activate, into proliferative matrix-producing cells that generate fibrosis [Van Waes, L. and Lieber, C. S., Gastroenterology. 1977; 73:646-650; Schuppan, D., et al., Semin. Liver Dis. 2001; 21:351-372]. Key markers of activated stellate cells include beta-PDGF receptor, matrix metalloproteinase-2 (MMP-2), intercellular adhesion molecule-1 (ICAM-1) and alpha smooth muscle actin (alpha SMA) [Van Waes and Lieber, 1977, id ibid.; Schuppman et al. (2001) id ibid.]. Stellate cell fibrogenesis reflects the activities of profibrotic cytokines including transforming growth factor beta (TGF beta 1) [Rojkind, M., et al., Gastroenterology 1979; 76:710-719] and connective tissue growth factor (CTGF) [Friedman, S. L., et al., J. Biol. Chem. 1989; 264:10756-10764] and anti-fibrotic cytokines that include interleukin-10 and interferon gamma (IFNγ) [McGuire, R. F., et al., Hepatology 1992; 15:989-997; Friedman, S. L., J. Biol. Chem. 2000; 275:2247-2250; Friedman, S. L., ed. The Hepatic Stellate Cell. Vol. 21. New York: Thieme; 2001; Friedman, S. L., Progress Liver Dis. 1996; 14:101-130; Gressner, A. M., J. Hepatol. 1995; 22:28-36]. An antifibrotic activity of IL-10 has been suggested by studies of cultured stellate cells in which neutralizing antibodies to IL-10 down-regulate matrix accumulation [McGuire, R. F., et al., 1992, id ibid.]; exogenous administration of IL-10 to experimental hepatitis animals results in attenuated fibrosis [Friedman, S. L., (1996) id ibid.; Gressner, A. M., (1995) id ibid.]; and increased fibrosis is often seen in IL-10 knockout mice in response to toxic injury [Friedman, S. L. and Arthur, M. J., J. Clin. Invest. 1989; 84:1780-1785; Gressner, A. M., et al., J. Hepatol. 1993; 19:117-132].

Recent studies have focused not only on cytokines, but also on the inflammatory cells from which they are secreted, including hepatic macrophages (Kupffer cells), natural killer (NK) cells and lymphocytes, including CD4+ T helper (Th) and CD8+ subsets [McGuire et al., (1992) id ibid.; Friedman, S. L. (2000) id ibid.]. The lymphocyte subsets can also be broadly divided into those that are either Th1 or Th2 predominant [Wang, S. C., et al., J. Biol. Chem. 1998; 273:302-308]. For example, IFN-gamma, a Th1 lymphocyte cytokine, has a potent antifibrotic activity [Wang et al. (1998) id ibid.; Winwood, P. J., et al., Hepatology 1995; 22:304-315]. C57BL/6 mice that exhibit increased level of interferon gamma producing Th1 cells have comparatively minimal fibrosis, whereas BALB/c mice that mainly develop a Th2 response generate a severe fibrosis in response to carbon tetrachloride [Wang et al. (1998) id ibid.; Yu, Q. and Stamenkovic, I., Genes Dev. 2000; 14:163-176; Nieto, N., et al., Hepatology 1999; 30:987-996]. Moreover, severe combined immunodeficient (SCID) mice lacking interferon gamma exhibit more pronounced fibrosis than wild-type animals [Winwood et al. (1995) id ibid.]. These data suggest that hepatic fibrosis is influenced by the responses of the different immune cell subsets, and that Th1/Th2 cytokine subsets can modulate the liver injury originated from a fibrotic response to toxin. Studies of this type have instigated the search for genetic loci that correlate with risk of fibrosis in mice and in humans, with the presumption that such loci may encode for genes that modulate the immunologic response [Nieto, N., et al., Hepatology 1999; 30:987-996; Svegliati-Baroni, G., et al., Liver 2001; 21:1-12].

Immune mediated regulation of human liver fibrosis is increasingly valued, and immunosuppression has been identified as an important arousing condition. For example, patients with human immunodeficiency virus (HIV) infection are at accelerated risk of fibrosis when co-infected with hepatitis C virus (HCV), independently of the liver injury extent [Bachem, M. G., et al., J. Clin. Chem. Clin. Biochem. 1989; 27:555-565; Casini, A., et al., Hepatology 1997; 25:361-367; Winnock, M., et al., J. Gastroenterol. Hepatol. 1995; 10:S43-S46]. Similarly, progression to cirrhosis, in liver transplanted patients (who require long-term immunosuppressive therapy) chronically infected with HCV, is greatly accelerated compared to those with native infection [Casini et al. (1997) id ibid.; Winnock et al. (1995) id ibid.], and a subset develop a rapidly progressive fibrogenic syndrome that may lead to recurrent cirrhosis within a year or less [Guler, M. L., et al., Science 1996; 271:984-987].

Fibrosis is a reversible scarring response that occurs in almost all patients with chronic liver injury. Ultimately hepatic fibrosis leads to cirrhosis, characterized by nodule formation and organ contraction. The causes of cirrhosis are multiple and include congenital, metabolic, inflammatory, and toxic liver disease. Examples including schistosomiasis, idiopathic portal fibrosis, alcohol, methotrexate, isoniazid, vitamin A, amiodarone, chronic HBV & HCV, Echinococcus, autoimmune chronic hepatitis, chronic passive congestion, Wilson's disease, genetic hemochromatosis, alpha 1-antitrypsin deficiency, carbohydrate metabolism disorders, primary biliary cirrhosis, secondary biliary cirrhosis, cystic fibrosis, biliary atresia/neonatal hepatitis, congenital biliary cysts, non-alcoholic steato-hepatitis and veno-occlusive disease. Taking all together; the target population of hepatic fibrosis accounts tens of millions of patients worldwide. Therefore, the development of effective antifibrotic therapies becomes crucial. These therapies are aimed at inhibiting the accumulation of activated hepatic stellate cells (HSCs) at the sites of liver injury and preventing the deposition of extracellular matrix. Although many of these approaches are effective in experimental models of liver fibrosis, their efficacy and safety in humans are still unknown. Thus far no drugs are approved as anti-fibrotic agents in humans.

The paradigm of stellate cell activation provides an important framework for defining sites/targets of antifibrotic therapy [for review see Bataller, R. and Brenner, D. A., Semin Liver Dis. 2001; 21:437-452]. These strategies include: (A) curing the primary disease to prevent injury; (B) reducing inflammation or the host response in order to avoid stimulating stellate cell activation; (C) directly downregulating stellate cell activation; (D) neutralizing proliferative, fibrogenic, contractile, and/or proinflammatory responses of stellate cells; (E) stimulating apoptosis of stellate cells; and (F) increasing the degradation of scar matrix, either by stimulating cells that produce matrix proteases, downregulating their inhibitors, or by direct administration of matrix proteases.

Liver fibrosis and cirrhosis from all etiologies are major causes for hepatocellular carcinoma (HCC). HCC is one of a group of neoplasms complicating mainly cirrhotic patients. Although palliative treatment such as surgical resection, chemoembolization, and intra-tumor alcohol injection have prolonged survival, in general, the prognosis remains poor for the majority of patients [Farmer D. G., et al. Ann. Surg. 1994; 219:236-247]. HBV associated HCC express HBsAg on their cell surface which in this particular situation, may serve as a tumor associated antigen [Shouval D., et al., Proc. Natl. Acad. Sci. USA 1988; 85:8276-8280].

CD56+ T cells and NK cells but not regular T cells purified from liver MNC cultured with cytokines showed potent cytotoxicities against HuH-7 HCC cells suggesting that a decreased number of CD56+ T cells and NK cells in cirrhotic livers may be related to their susceptibility to HCC [Kawarabayashi, N., et al., Hepatology. 2000; 32(5):962-9]. NK cell activity was decreased significantly in HCC patients compared with control groups suggesting that the preoperative NK cell activity will help predict recurrence and prognosis after hepatectomy in patients with HCC [Taketomi A, et al. Cancer. 1998; 83(1):58-63].

Interleukin-2 (IL-2) is well accepted as a T cell growth factor. IL-2 is a promising immunotherapeutic agent for the treatment of metastatic melanoma, acute myelogenous leukemia, and metastatic renal cell carcinoma. While high-dose IL-2 regimens have shown clinical benefit in the treatment of melanoma and renal cell carcinoma, serious dose-limiting toxicities have limited their clinical use in a broader group of patients. Low-dose IL-2 therapy has produced disappointing clinical response rates in melanoma. While the response rates to low-dose IL-2 have been better in renal cell carcinoma, the quality of these responses relative to those seen with high-dose IL-2 therapy remains a concern. The addition of IL-2 to chemotherapeutic regimens (biochemotherapy) has been associated with overall response rates of up to 60% in patients with metastatic melanoma, but this has yet to be translated into a confirmed improvement in survival. It remains to be determined whether further modifications of IL-2-based regimens or the addition of newer agents to IL-2 will produce a better anti-tumor response and improve survival.

IL-2 has been considered an active and well-tolerated treatment for unresectable HCC. IL-2 treatment of renal cell carcinoma and melanoma, was initially associated with treatment-related mortality because its highly toxicity. Although, in the appropriate setting IL-2 can be administered safely. Low-dose IL-2 can be considered an active and well-tolerated treatment for unresectable hepatocellular carcinoma. Following prolonged, ultra-low-dose (1 MIU/d until progression), subcutaneous IL-2 in a series of 18 patients (14 men and 4 women, median age 66 years, range 49-82 years) with advanced histologically proven HCC on liver cirrhosis, a median follow-up time of 19.5 months was reported. Two complete responses (defined as tumor resolution) were observed (11.1%), lasting 35 and 46 months, respectively, and one partial response (5.5%) were recorded (overall response rate: 16.6%; 95% CI: 0-33.8%). Thirteen patients (72.3%; 95% CI: 61.6-82.7) had stable disease lasting at least 4 months; 1 of these patients obtained a complete response on lung metastases. Median time to progression was 15.3 months (95% CI: 10-33). Median overall survival was 24.5 months (95% CI: 1243). Two patients (11.1%) progressed during therapy. Toxicity was only local (usually pain and pomphus in the site of injection) [Palmieri, G., et al. Am. J. Clin. Oncol. 2002; 25(3):224-6]. Ex vivo stimulation of tumor infiltrating lymphocytes (TILs) with interleukin-2 has been therapeutically used in some cancer patients with great success. Killing activity of tumor infiltrating lymphocytes derived from 6 patients with hepatocellular carcinoma (HCC) was augmented using autologous monocytes derived dendritic cells (DC). Autologous dendritic cells (from the same patient) were generated from CD14+ monocytes cultured for 6 days in the presence of granulocyte macrophage colony-stimulating factor (GM-CSF) and interleukin-4 (IL-4). Those professional antigen presenting cells were pulsed with whole autologous hepatoma tumor lysates (pDC). TILs were cocultured with pDC or unpulsed DC. The cytotoxic potency of TILs was estimated by their ability to lyse the tumor cell targets K652, Daudi cell lines and allogeneic HCC cells in a standard cytotoxic assay. Tumor cells targets cultured in vitro, were poorly lysed by tumor infiltrating lymphocytes indicating T-cell hyporesponsiveness. In contrast, the killing activity of HCC derived TILs against Daudi (9.15%+/−7.5) and allogeneic HCC tumor target (18.2%+/−9.2) could be significantly augmented when stimulated with pDC (Daudi: 38%+/−6.8 and allogeneic HCC: 55%+/−10). The killing activity of TILs against K562 was unaffected by pDC. Thus, the low cytotoxic activity profile of HCC derived TILs in vitro can be increased by tumor lysate pulsed dendritic cells and may therefore be more effective in vivo when used for adoptive immunotherapy. [Friedl, J., et al. Cancer Biother Radiopharm. 2000; 15: 477-86].

Inflammatory bowel diseases (IBDs) are common immune-mediated disorders of the gastrointestinal tract. An imbalance between Th1 proinflammatory and Th2 anti-inflammatory subtypes of immune responses plays a role in the pathogenesis of these disorders [Podolsky D K. New Engl J Med. 1991; 325: 928-935, Mizoguchi A., et al. J Exp Med 1996; 183: 847-856, Adorini L., et al. Immunol. Today 1997; 18: 209-211]. In both experimental colitis, and in patients with IBD, the disease is a Th1-mediated immune disorder, resulting in a life-long inflammatory response against the colon. Secretion of proinflammatory cytokines such as IFN-gamma has been described [Strober W., et al. Immunol Today. 1997; 18: 61-64]. Anti-inflammatory cytokines such as IL-10 down-regulate the proinflammatory effects of Th1-mediated cytokines, thereby alleviating the disease [Neurath M F., et al. J Exp Med. 1996; 183: 2605-2616, Madsen K L., et al. Gastroenterology. 1997; 113: 151-159, Van Deventer Sander J., et al. Gastroenterology. 1997; 113: 383-389].

The pathogenesis of IBD involves exposure of specific bowel mucosa epitopes as a consequence of a toxic, infectious, or immune-mediated effect [Hibi S., et al. Clin Exp Immunol. 1983; 54: 163-168, Das K M., et al. Gastroenterology. 1990; 98: 464-469, Podolsky D K. New Engl J Med. 1991; 325: 928-935, Dasgupta A., et al. Gut. 1994; 35: 1712-1717, Neurath M F., et al. J Exp Med. 1995; 182: 1281-1290]. These cryptic antigens incite an active autoimmune inflammatory response [Takahashi F., et al. J Clin Invest. 1985; 76: 311-318, Z'graggen K., et al. Gastroenterology. 1997; 113: 808-816]. Both in humans with IBD and in animals with TNBS-induced experimental colitis, the disease is a Th1-type immune-mediated disorder [Mizoguchi A., et al. J Exp Med. 1996; 183: 847-856, Neurath M F., et al. J Exp Med. 1995; 182: 1281-1290]. Stimulated cells in the inflamed mucosa produce increased amounts of IFN-gamma and IL-2 and reduced amounts of IL-4, thereby attracting inflammatory cells and disrupting mucosal integrity. In contrast, anti-inflammatory cytokines such as IL-10 down-regulate the proinflammatory effects of Th1 cytokines and may alleviate the disease [Madsen K L., et al. Gastroenterology. 1997; 113: 151-159].

Glatiramer acetate (Copaxone®) is a synthetic copolymer composed of a random mixture of four amino acids capable of modifying the autoimmune response against the CNS characteristic of relapsing-remitting multiple sclerosis (RRMS). This autoimmune reaction leads to inflammation of the CNS, demyelination and finally axonal loss. In three randomised, double-blind trials in patients with RRMS, subcutaneous administration of glatiramer acetate (20 mg/day) has been shown to be significantly effective by mean of relapse rate, proportion of relapse-free patients and number of gadolinium-enhancing lesions seen on magnetic resonance imaging [MRI] scans). Glatiramer acetate has also been shown to significantly decrease disease activity and burden of disease, as assessed in the European/Canadian study using a range of MRI measures. Patients with RRMS treated with glatiramer acetate were significantly more likely to experience disability attenuation and their overall disability status significantly improved. Glatiramer acetate is generally well tolerated; the most commonly reported treatment-related adverse events were localised injection-site reactions and transient post-injection systemic reactions, both reactions generally mild and self limiting. Glatiramer acetate is not associated with the influenza-like syndrome or neutralizing antibodies that are reported in patients treated with interferon-beta for RRMS. Based on available data and current management guidelines, glatiramer acetate is a valuable first-line treatment option for patients with RRMS. [Simpson, D., et al., 2002; 16(12):825-850; BioDrugs 2003; 17(3):207-10.]

Glatiramer acetate (Copaxone, Teva Ltd.) was reported to promote Th2 CD4 cell development and increase IL-10 production through modulation of dendritic cells in experimental autoimmune encephalomyelitis [Copaxone: Vieira, P. L., et al., J. Immunol. 2003].

Glatiramer acetate prevents graft-versus-host disease and interferes in various manifestations of immune rejection. In two transplantation systems for skin and thyroid grafting assays, Glatiramer acetate treatment prolonged skin graft survival and inhibited the functional deterioration of thyroid grafts. Glatiramer acetate inhibited the proliferation of graft-specific T cell lines, as well as their interleukin-2 and interferon-gamma secretion, when incubated in vitro with the stimulating allogeneic cells. Glatiramer acetate treatment inhibited the Th1 response to graft and induced a Th2 cytokines secretion in response to both Glatiramer acetate and graft cells, leading to improved survival and function of the transplanted grafts [Aharoni, R., et al., Transplantation. 2001].

In search for agents which may be effective in the treatment of hepatic fibrosis, which is an object of the present invention, the inventors investigated the impact of Copaxone on mouse hepatic fibrogenesis in vivo.

Furthermore, it is suggested, and this is another object of the invention, that anti-fibrotic immune modulation by Copaxone, optionally in combination with IL-2, can serve also as anti fibrosis-associated HCC.

In a different aspect, Copaxone administration to patients suffering from inflammatory bowel diseases might result in a new treatment strategy.

These, and other objects of the invention will become apparent as the description proceeds.

### SUMMARY OF THE INVENTION

The present invention relates to a method for the treatment of hepatic fibrosis comprising administering to a subject in need thereof a therapeutically effective amount of an immuno-modulatory agent.

More particularly, the invention relates to a method for the treatment of hepatic fibrosis comprising administering to a subject in need thereof an immuno-modulatory agent which elevates the CD4:CD8 ratio and/or increases the number of NK cells and/or elevates the NK aKIR:NK iKIR ratio in hepatic tissue.

A preferred immuno-modulatory agent is glatiramer acetate.

The method of the invention is particularly intended for the treatment of human.

The invention further relates to use of an immuno-modulatory agent in the preparation of a pharmaceutical composition for the treatment of hepatic fibrosis, particularly an immuno-modulatory agent which elevates the CD4:CD8 ratio and/or increases the number of NK cells and/or elevates the NK aKIR:NK iKIR ratio in hepatic tissue.

In this aspect, the invention preferably relates to the use of glatiramer acetate in the preparation of a pharmaceutical composition for the treatment of hepatic fibrosis.

Still further, the invention relates to an immuno-modulatory agent, particularly an agent which elevates the CD4:CD8 ratio and/or increases the number of NK cells and/or elevates the NK aKIR:NK iKIR ratio in hepatic tissue, and most preferably glatiramer acetate, for use in the treatment of hepatic fibrosis.

In yet another aspect, the invention relates to a method for the treatment of hepatic fibrosis by elevating CD4:CD8 ratio and/or increasing the number of NK cells and/or elevating NK aKIR:NK iKIR ratio in hepatic tissue comprising administering to a subject in need of such treatment an immuno-modulatory agent that elevates the CD4:CD8 ratio and/or increases the number of NK cells and/or elevates the NK aKIR:NK iKIR ratio in hepatic tissue. A preferred immuno-modulatory agent to be administered is glatiramer acetate.

The immuno-modulatory agent, preferably glatiramer acetate, is used for elevating CD4:CD8 ratio and/or increasing NK cells number and/or elevating NK aKIR:NK iKIR ratio in fibrotic liver tissue, thereby reducing hepatic fibrosis.

In a further aspect, the invention relates to a method for screening for an immuno-modulatory agent which is useful in the treatment of hepatic fibrosis, comprising the steps of: (a) providing a test agent; (b) providing a fibrosis-induced model animal and a non-fibrotic model animal; (c) administering said test agent to said fibrotic and non-fibrotic animals; (d) obtaining samples of hepatic tissue from said animals; (e) measuring at least one of the CD4:CD8 ratio, the number of NK cells, NK aKIR:NK iKIR ratio, area of the fibrotic tissue in said sample and at least one accepted fibrosis parameter; and (f) comparing the results obtained for the sample obtained from the fibrosis-induced animal with the corresponding results obtained for the non-fibrotic animal; whereby elevation of CD4:CD8 ratio, and/or increased number of NK cells, and/or elevated NK aKIR:NK iKIR ratio, and/or reduced area of the fibrotic tissue and/or decrease of said accepted fibrosis parameter indicates that said test agent is useful in the treatment of hepatic fibrosis.

Accepted liver injury parameters may be AST, ALT, Ishak injury score. Fibrosis parameters, however, are Ishak fibrosis score, computerized Bioquant® quantitation and alfa smooth muscle actin assessment using Western blotting analysis.

In another aspect, the invention relates to a method for the treatment of any one of hepatic cellular carcinomas and inflammatory bowel diseases comprising administering to a subject in need thereof a therapeutically effective amount of at least one immuno-modulatory agent that elevates the CD4:CD8 ratio and/or increases the number of NK cells and/or elevates the NK aKIR:NK iKIR ratio in hepatic or bowel tissue.

Preferably, the immuno-modulatory agent for the treatment of human subjects, is glatiramer acetate, optionally in combination with IL-2.

More specifically, the immuno-modulatory agent, desirably glatiramer acetate possibly in combination with IL-2, which elevates the CD4:CD8 ratio and/or increases the number of NK cells and/or elevates NK aKIR:NK iKIR ratio in hepatic or bowel tissue, may be used in the preparation of a pharmaceutical composition for the treatment of any one of hepatic cellular carcinomas and inflammatory bowel diseases.

### BRIEF DESCRIPTION OF THE FIGURES

FIG. 1: AST and ALT serum level in CCl4-fibrosis induced mice AST and ALT serum level were measured in CCl4-fibrosis induced mouse model in order to assess the hepatic injury and estimate the treatment efficacy.

Abbreviations: AST: Aspartate aminotransferase; ALT: Alanine transaminase; Nai.: naïve; Copax.: Copaxone; Linom.: Linomycin; Treat.: treatment; u/l: units/liter.

FIG. 2: Hepatic injury and fibrosis scores in CCl4-fibrosis induced mice

Pathological and Bioquant® analysis showed significant increase of hepatic injury and hepatic fibrosis in all the groups treated with CCl4 when compared to naïve animals. Copaxone- and Linomycin-treated groups showed a significant reduction of fibrosis compared to the CCl4 fibrosis-induced untreated group (1 values were <0.001).

Abbreviations: Nai.: naïve; Copax.: Copaxone; Linom.: Linomycin; Hep. Inj. Sc.: hepatic injury score; Inj. Sc.: injury score; Fibr. Sc.: fibrosis score; % fibr. Ar.: percentage of hepatic fibrotic area.

FIG. 3a-b: Liver tissue fibrosis pathology in CCl4-fibrosis induced mice Sirius Red F3B liver sections.

FIG. 3a: Liver section from a naïve mouse.

FIG. 3b: Liver section from a CCl4 fibrosis-induced mouse treated with Copaxone.

FIG. 4: Liver tissue fibrosis estimated by % of collagen in hepatic lesion area in CCl4-fibrosis induced mice

Abbreviations: Nai.: naïve; Copax.: Copaxone; Linom.: Linomycin; % coll. Ar.: percentage of collagen in hepatic fibrotic area.

FIG. 5: Liver tissue fibrosis estimated by αSMA expression in CCl4-fibrosis induced mice

Western blot analysis of liver tissue samples from fibrosos and control mice using anti-αSMA antibodies.

Abbreviations: Nai.: naïve; Copax.: Copaxone; Linom.: Linomycin; αSMA: alpha smooth muscle actin; KD: kilodaltons.

FIG. 6: Spleen CD4 and CD8 cell populations in CCl4-fibrosis induced mice

Spleen CD4 and CD8 cell counts expressed relatively to the splenic lymphocyte population.

Abbreviations: Nai.: naïve; Copax.: Copaxone; Linom.: Linomycin; % tot. lymp.: percentage of splenic lymphocyte population.

FIG. 7: Spleen CD4/CD8 cell populations ratio in CCl4-fibrosis induced mice

Abbreviations: Nai.: naïve; Copax.: Copaxone; Linom.: Linomycin; Treat.: treatment; Rat.: ratio.

FIG. 8: Spleen NK cell population in CCl4-fibrosis induced mice

NK cell were estimated relatively to the splenic CD45 cell population.

Abbreviations: Nai.: naïve; Copax.: Copaxone; Linom.: Linomycin; Treat.: treatment; % CD45: percentage of CD45 cell population.

FIG. 9: Spleen NK aKIR/NK iKIR ratio in CCl4-fibrosis induced mice

aKIR:iKIR ratio was calculated from results of separated readings for aKIAR, iKIR and total NK cells.

Abbreviations: Nai.: naïve; Copax.: Copaxone; Linom.: Linomycin; Treat.: treatment; NK aKIR:NK iKIR Rat.: NK aKIR:NK iKIR ratio.

FIG. 10: Liver CD4 and CD8 cell populations in CCl4-fibrosis induced mice

Hepatic CD4 and CD8 cell counts expressed relatively to the hepatic lymphocyte population.

Abbreviations: Nai.: naïve; Copax.: Copaxone; Linom.: Linomycin; % lymp.: percentage of hepatic lymphocyte population.

FIG. 11: Liver CD4/CD8 cell populations ratio in CCl4-fibrosis induced mice

Abbreviations: Nai.: naïve; Copax.: Copaxone; Treat.: treatment; Rat.: ratio.

FIG. 12: Liver NK cell population in CCl4-fibrosis induced mice

NK cell were estimated relatively to the hepatic CD45 cell population.

Abbreviations: Copax.: Copaxone; % CD45: percentage of hepatic CD45 cell population.

FIG. 13a-b: Colitis score in colitis-induced mice

Colitis score was estimated macroscopically and microscopically in colitis-induced mice and naïve controls and compared to mice treated or untreated with Copaxone.

FIG. 13a: Macroscopic assessment of tissue injury in colitis-induced mice.

FIG. 13b: Microscopic assessment of tissue injury in colitis-induced mice.

Abbreviations: Col.: colitis; Copax.: Copaxone; Nai.: naïve.

FIG. 14a-d: Colon pathology in colitis-induced mice

Hematoxylin and eosin rectosigmoid colon sections.

FIG. 14a: Naïve mouse

FIG. 14b: Naïve mouse treated with Copaxone

FIG. 14c: Colitis-induced mouse

FIG. 14d: Colitis-induced mouse treated with Copaxone

FIG. 15: Spleen CD3, CD4 and CD8 cell populations in colitis-induced mice

Spleen CD3, CD4 and CD8 cell counts expressed relatively to the splenic lymphocyte population.

Abbreviations: Col.: colitis; Copax.: Copaxone; Nai.: naïve; % CD45: percentage of splenic CD45 cell population.

FIG. 16: CD4 IL-4 and CD4 IFN-γ secreting cell subpopulations in colitis-induced mice

CD4 IL-4 and CD4 IFN-γ secreting cell subpopulations were estimated relatively to whole CD4 cell population.

Abbreviations: Col.: colitis; Copax.: Copaxone; Nai.: naïve.

FIG. 17: APC population in colitis-induced mice

Splenic antigen presenting cells calculated relatively to splenic CD45 cell population.

Abbreviations: Col.: colitis; Copax.: Copaxone; Nai.: naïve.

FIG. 18: Serum level of IL-4, IL-10 and IFN-γ cytokines in colitis-induced mice

Abbreviations: Col.: colitis; Copax.: Copaxone; Nai.: naïve; pg/ml: pictogram/milliliter.

### ABBREVIATIONS

The following abbreviations are used in this application:

iKIR Inhibitory killing immunoglobulin receptor

aKIR Activation killing immunoglobulin receptor

NK Natural killer cell

HSC Hepatic stellate cell

I.P. Intraperitoneal

AST Aspartate aminotransferase

ALT Alanine transaminase

ECM Extracellular matrix

HCC Hepatic cellular carcinoma

HSC Hepatic stellate cell

### DETAILED DESCRIPTION OF THE INVENTION

As will be shown in the following Examples, in the experimental models used by the inventors, natural killer (NK) cells have anti-fibrotic activity via their activation combined with increased killing of stimulated HSCs. Thus, in the experimental models used by the inventors, NK cells have anti-fibrotic activity via their activation combined with increased killing of activated HSCs. In vivo they may occur opposing effects: loss of CD4, which favors fibrogenesis and NK activation, which is anti-fibrogenic. These findings significantly extend the understanding of the immune system involvement in the fibrosis process and point to manipulation of CD4, CD8 and NK subsets as potential options for a therapeutic approach to modulate fibrosis.

With the aim of finding agents which can be used in the treatment and/or prevention of liver fibrosis, the inventors proposed that elevating the ratio C4:CD8 in fibrotic hepatic tissue, thereby increasing the number of NK cells, may inhibit the fibrosis.

In the present work, the inventors have found that treatment of fibrosis-induced mice with immuno-modulatory agents, such as glatiramer acetate, increased the CD4:CD8 ratio and improved in their condition as measured by several scores.

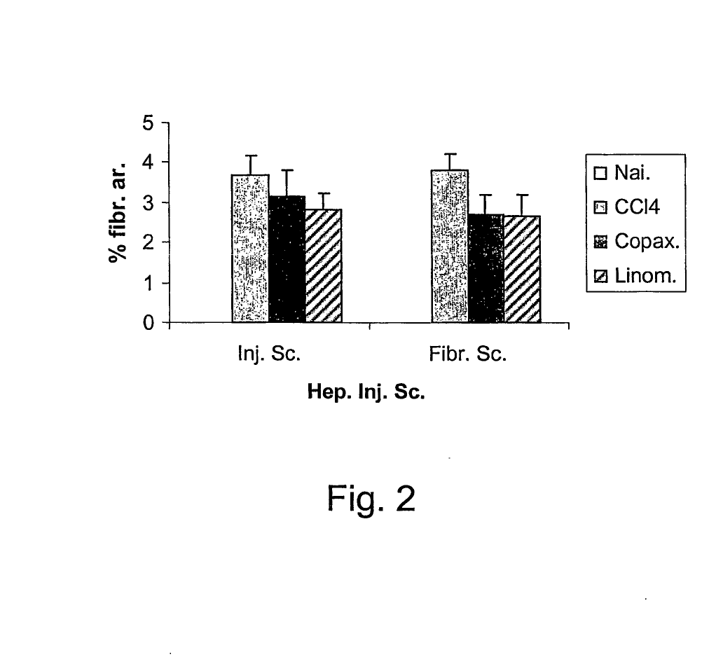

As shown in the following Examples, and particularly FIGS. 2, 4 and 5 fibrosis was significantly increased in all the CCl4 induced groups, as measured by the Ishak liver fibrosis scoring and computerized Bioquant® analysis, which are accepted parameters for the evaluation of the fibrosis extent. Animals treated with glatiramer acetate showed significantly lower scores. Also the Ishak liver injury scoring, although it was significantly increased following fibrosis induction in all fibrotic groups, it was significantly lower in the glatiramer acetate and linomycin treated groups

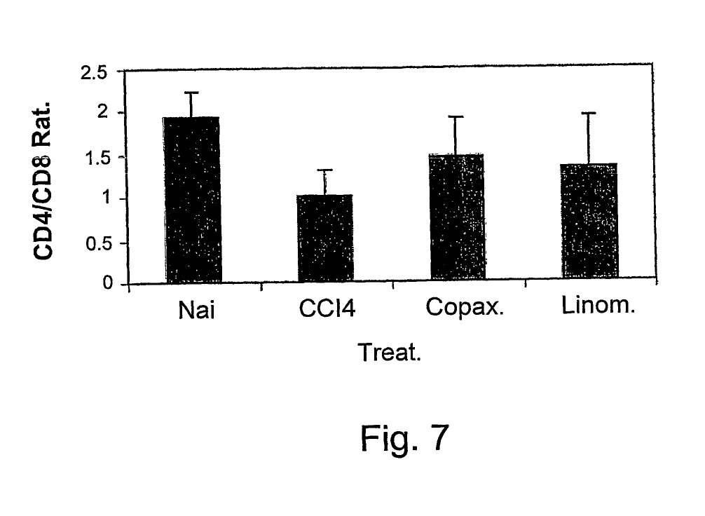

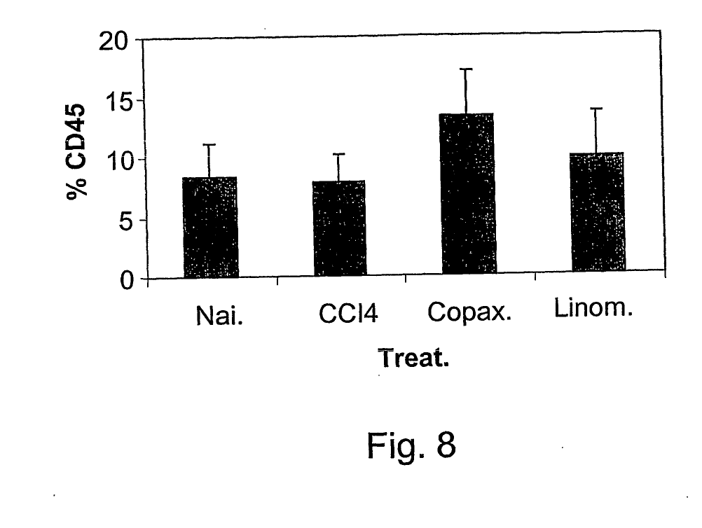

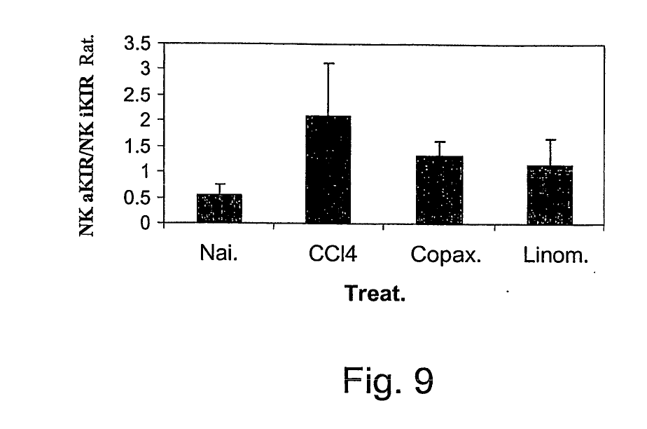

As may be seen in FIG. 7 CD4:CD8 ratio was significantly elevated in the glatiramer acetate treated group when compared to the control group (CCl4-induced fibrosis untreated group). The total number of NK cells was also elevated in the treated groups (glatiramer acetate and linolin groups) (FIG. 8). Moreover, a prominent change in the NK aKIR:NK iKIR ratio was observed in the treated groups (FIG. 9).

Thus, the inventors have shown that immuno-modulatory agents, and particularly glatiramer acetate (Copaxone), had a significant anti-fibrotic effect in the used animal model. Without being bound by theory, it is suggested that this effect was mediated by increasing the CD4:CD8 ratio, which increased the total number of NK cells. Those NK cells were stimulated and activated against the activated stellate cells and thus decreased fibrosis.

Thus, the present invention relates to the use of immuno-modulatory agents, particularly glatiramer acetate, in the treatment of hepatic fibrosis. Furthermore, the invention provides methods for the treatment of hepatic fibrosis by administering to a patient in need a therapeutically effective amount of an immuno-modulatory agent in accordance with the invention, and to compositions comprising these agents for the treatment of the disease.

Particularly preferred immuno-modulatory agents in accordance with the invention are those capable of elevating CD4:CD8 ratio and/or increasing the number of NK cells and/or elevating NK aKIR:NK iKIR ratio in hepatic tissue, particularly fibrotic hepatic tissue.

Most preferred agent is glatiramer acetate.

The pharmaceutical compositions of the invention comprise as the active ingredient the immuno-modulatory agent of the invention, particularly glatiramer acetate, and may optionally further comprise additional therapeutic agents and/or pharmaceutically acceptable carriers, excipients and/or diluents.

The preparation of pharmaceutical compositions is well known in the art and has been described in many articles and textbooks, see e.g., Remington's Pharmaceutical Sciences, Gennaro A. R. ed., Mack Publishing Co., Easton, Pa., 1990, and especially pp. 1521-1712 therein, fully incorporated herein by reference.

The pharmaceutical composition of the invention can be administered and dosed in accordance with good medical practice. Administration may be carried out in various ways, including intravenous, intramuscular or subcutaneous injection. However, other methods of administration such as oral administration are also possible.

The composition of the invention may comprise the active substance in free form and be administered directly to the subject to be treated. Alternatively, depending on the size of the active molecule, it may be desirable to conjugate it to a carrier prior to administration. Therapeutic formulations may be administered in any conventional dosage formulation. Formulations typically comprise at least one active ingredient, as defined above, together with one or more acceptable carriers thereof.

Each carrier should be both pharmaceutically and physiologically acceptable in the sense of being compatible with the other ingredients and not injurious to the patient. Formulations include those suitable for oral, rectal, nasal, or parenteral (including subcutaneous, intramuscular, intraperitoneal (IP), intravenous (IV) and intradermal) administration. The formulations may conveniently be presented in unit dosage form and may be prepared by any methods well known in the art of pharmacy. The nature, availability and sources, and the administration of all such compounds including the effective amounts necessary to produce desirable effects in a subject are well known in the art and need not be further described herein.

More specifically, the active agents of the invention or compositions comprising the same, may be administered by a route selected from oral, intravenous, parenteral, transdermal, subcutaneous, intravaginal, intranasal, mucosal, sublingual, topical and rectal administration and any combinations thereof. Preferably, these immuno-modulatory agents or compositions are IV or IP injected.

The pharmaceutical forms suitable for injection use include sterile aqueous solutions or dispersions and sterile powders for the extemporaneous preparation of sterile injectable solutions or dispersions. In all cases the form must be sterile and must be fluid to the extent that easy syringeability exists. The compositions must be stable under the conditions of manufacture and storage and must be preserved against the contaminating action of microorganisms, such as bacteria and fungi.

The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above.

In the case of sterile powders for the preparation of the sterile injectable solutions, the preferred method of preparation are vacuum-drying and freeze drying techniques which yield a powder of the active ingredient plus any additional desired ingredient from a previously sterile-filtered solution thereof.

The pharmaceutical compositions of the invention generally comprise a buffering agent, an agent that adjusts the osmolarity thereof, and optionally, one or more pharmaceutically acceptable carriers, excipients and/or additives as known in the art. Supplementary active ingredients can also be incorporated into the compositions. The carrier can be solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants.

As used herein “pharmaceutically acceptable carrier” includes any and all solvents, dispersion media, coatings, antibacterial and antifungal agents and the like. The use of such media and agents for pharmaceutical active substances is well known in the art. Except as any conventional media or agent is incompatible with the active ingredient, its use in the therapeutic composition is contemplated.

Dose will depend on weight, age, sex, severity of the disease and tolerability, and will be determined by the attending physician. Preferred doses in humans are from about 15 to about 20 mg subcutaneous injections once daily for 2 years, or from about 5 to about 50 mg orally per day, and most preferably 20 mg subcutaneous injections once daily for 2 years.

The invention also relates to a method for the treatment or prevention of hepatic disorder, comprising administering the agent of the invention or a pharmaceutical composition of the invention or of any of the preferred embodiments thereof, to a patient in need thereof.

The invention also relates to a method of administering to a patient in need of such treatment a therapeutic agent for treatment of a disorder or disease of the liver, comprising the steps of administering to said patient the active agent of the invention and said therapeutic agent. The administration of the therapeutic agent may be simultaneous with the administration of that of the immuno-modulatory agent of the invention, or preceding or following the same.

The inventors' findings can be used to screen for therapeutic agents that can be used in the treatment of hepatic fibrosis. Thus, in a further aspect, the invention relates to a method for screening for an immuno-modulatory agent which is useful in the treatment of hepatic fibrosis, comprising the steps of (a) providing a test agent; (b) providing a fibrosis-induced model animal and a non-fibrotic model animal; (c) administering said test agent to said fibrotic and non-fibrotic animals; (d) obtaining samples of hepatic tissue from said animals; (e) measuring at least one of the CD4:CD8 ratio, the number of NK cells, NK aKIR:NK iKIR ratio, area of the fibrotic tissue in said sample and an accepted fibrosis parameter; and (f) comparing the results obtained for the sample obtained from the fibrosis-induced animal with the corresponding results obtained for the non-fibrotic animal; whereby elevation of CD4:CD8 ratio, and/or increased number of NK cells, and/or elevated NK aKIR:NK iKIR ratio, and/or reduced area of the fibrotic tissue and/or decrease of said accepted fibrosis parameter indicates that said test agent is useful in the treatment of hepatic fibrosis.

Preferred accepted fibrosis parameters may be Ishak liver fibrosis scoring, computerized Bioquant® analysis, hydroxyproline (HP), but any suitable end point, indicative of amelioration of the fibrosis can be used in the screening method of the invention. AST, ALT, and Ishak injury score are accepted as liver injury parameters.

In the screening method of the invention, standard values for the hepatic fibrosis scores may be used for comparison, instead of or in addition to the said control, non-fibrotic animals.

The screening method of the invention may also be used for the identification of immuno-modulatory agents which can be advantageous for HCC and IBD treatment when using the suitable animal model and clinical parameters.

Chronic active hepatitis which is associated with ongoing liver cell injury, dramatically increases the risk of developing hepatocellular carcinoma (HCC). The chronic inflammatory process, accompanied by liver cell death and regeneration, may ultimately lead to transforming mutations in hepatocytes. Integration of viral DNA, a common event in HCC cells, may disturb host cell gene regulation and lead to malignant degeneration. HBV infection can be overcome in mice by direct microinjection of HBV genes. When hepatitis B surface antigen (HBsAg) gene integrates, and is expressed at high levels, liver cell injury and HCC develop. Also, the hepatitis B virus X protein (HBx) gene alone, when expressed at high levels can cause liver cancer. Therefore, different HBV transgenic mice may be used as an animal model for HCC. HCV transgenic mice is also a suitable model for HCC.

Hepatic damage can be assessed by scoring the following pathological parameters: portal vein invasion, intrahepatic metastasis, hepatic vein invasion, serosal invasion, absence of tumor capsule, or presence of capsular invasion. Otherwise, the CLIP score which includes the Child-Pugh stage, tumor morphology and extension, serum alfa-fetoprotein (AFP) levels, and portal vein thrombosis parameters, can be considered.

In connection with IBD, the experimental colitis animal model induced by intrarectal administration of trinitrobenzene sulfonic acid (TNBS) should be preferred. Colonic damage is manifested by the difference in the extent of cell death, tissue disorganization, and edema. These parameters can be measured after tissue fixation and staining with H&E. Histological damage score can be assess, for example, by the method of crypt scoring by Cooper [Cooper et al. Lab. Invest. 1993; 69: 238-249].

In, vitro screening methods are also contemplated within the scope of the present invention.

In a further aspect the invention relates to the use of glatiramer acetate, optionally in combination with IL-2, in the treatment of HCC. As mentioned above, liver fibrosis and cirrhosis from all etiologies is a major cause for hepatocellular carcinoma (HCC). As shown in the following Examples, decreased CD4/CD8 ratio favours fibrogenicity, while NK cells have anti-fibrotic effect by increased apoptosis of activated HSCs. Following glatiramer acetate treatment, NK cells and CD4/CD8 ratio increased in animal models, accompanied with decreasing the fibrosis, and as IL-2 treatment in HIV patients increases CD4 counts, and as NK cells were suggested to have an anti-HCC effect, it is proposed that the anti-tumor immune response against fibrosis-associated liver tumors can be improved by increasing NK cells induction and the CD4/CD8 ratios. Hepatitis B virus (HBV)-associated HCC expresses HBsAg on its cell surface and may serve as a tumor-associated antigen. Details of the suggested treatment are presented in the following Examples. Glatiramer acetate, optionally in combination with IL-2, may have a good anti-tumoral effect against the HCC via increasing the stimulation and absolute number of NK cells and via the increase of the CD4/CD8 ratio. The effect of each compound may be tested for a possible synergistic effect of treatment with their combination, and in order to verify whether their effect is directly anti-tumoral or whether it is mediated by anti-fibrotic effect. As both compounds are in the clinical human use in other indications, and safety and tolerability are well accepted, the results of this study might open a new therapeutic approach in the HCC particularly and many of other tumors generally.

In this aspect of the invention, also contemplated are pharmaceutical compositions comprising glatiramer acetate, optionally additional immune modulating agents such as, but not limited to, IL-2, for the treatment and/or prevention of HCC. Methods of treatment of HCC with glatiramer acetate, alone or in combination with IL-2 are also encompassed.

In a further aspect the invention relates to the use of glatiramer acetate, optionally in combination with IL-2, in the treatment of inflammatory bowel disease, mainly ulcerative colitis and Crohn's disease. As inflammatory bowel disease is mainly inflammatory injury of colon and as Glatiramer acetate reduced liver injury of the fibrosis model. Glatiramer acetate, and IL-2, alone or in combination, may have a good anti-inflammatory effect against the inflammatory bowel disease via increasing the stimulation and absolute number of NK cells and via the increase of the CD4/CD8 ratio. The effect of each compound may be tested for a possible synergistic effect of treatment with their combination, and in order to verify whether their effect is directly anti-inflammatory effect. As both compounds are in the clinical human use in other indications, and safety and tolerability are well accepted, the results of this study might open a new therapeutic approach in the inflammatory bowel disease particularly and many of other inflammatory diseases generally.

In this aspect of the invention, also contemplated are pharmaceutical compositions comprising glatiramer acetate, optionally additional immune modulating agents such as, but not limited to, IL-2, for the treatment and/or prevention of inflammatory bowel disease. Methods of treatment of inflammatory bowel disease with glatiramer acetate, alone or in combination with IL-2 are also encompassed.

Throughout this application, various publications are cited. These publications, including publications cited therein, are fully incorporated herein by reference.

Disclosed and described, it is to be understood that this invention is not limited to the particular examples, process steps, and materials disclosed herein as such process steps and materials may vary somewhat. It is also to be understood that the terminology used herein is used for the purpose of describing particular embodiments only and not intended to be limiting since the scope of the present invention will be limited only by the appended claims and equivalents thereof.

It must be noted that, as used in this specification and the appended claims, the singular forms “a”, “an” and “the” include plural referents unless the content clearly dictates otherwise.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The following Examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the scope of the invention.

### EXAMPLES

Materials

Carbon tetrachloride (CCl4; Sigma, C-5331), recombinant IL-2 (rIL-2), glatiramer acetate (Copaxone, Teva Ltd.). 2,4,6-Trinitrobenzenesulfonic acid (TNBS, Sigma Diagnostics, St. Louis, Mo.).

Animals: BALB/c mice from 3 groups were used: A) wild-type (WT), B) severe combined immunodeficiency (SCID) mice (lacking B and T cells) and C) SCID Beige Backing B, T and NK cells), which were compared to a fourth untreated WT group. C57BL/6 mice (wild type and SCID) were also used. Animals received care according to National Institutes of Health guidelines.

Experimental Design:

(I) The Role of NK Cells in Hepatic Fibrosis:

In this study, hepatic fibrosis was induced by intra-peritoneal Carbon Tetrachloride (CCl4) administration for 4 weeks in 8 week-old male BALB/c mice from 3 groups: A) wild-type (WT), B) severe combined immunodeficiency (SCID) mice (lacking B and T cells) and C) SCID Beige (lacking B, T and NK cells), which were compared to a fourth untreated WT group. Hepatic fibrosis was evaluated by Western blot for alpha-smooth muscle actin (α-SMA) from liver extracts, and by analyzing 36 Sirius Red stained histological liver sections per animal with Bioquant® morphometry system. Splenocytes, intrahepatic lymphocytes (IHL) and hepatic stellate cells (HSC) were isolated for FACS analysis from untreated WT and CCl₄ fibrotic WT animals.

(II) The Immune Therapy of Hepatic Fibrosis:

Hepatic fibrosis was induced by IP CCl₄ administration for 6 weeks in 8 week-old male wild-type C57Bl/6 mice. Within the last 2 weeks animals were also treated with either Copaxone 200 microgram/day I.P (Group A), Linomycin given by drinking water 1 mg/ml (Group B) or normal saline (group C), and were compared to naïve mice (Group D). Eight animals were included in each group. Hepatic fibrosis was evaluated by Ishak Knodell score [Knodell, R. G. et al., Hepatology 1981; 1(5):431-5], hydroxyproline and by analyzing 36 Sirius Red stained histological liver sections per animal with Bioquant® morphometry system. Splenocytes were isolated for FACS analysis from all animal groups. The correlation between hepatic fibrosis and CD4, CD8, NK cells, iKIR and aKIR expression on splenocytes was assessed.

(III) The Immune Therapy of Hepato Cellular Carcinoma:

Following splenocytes reconstitution in the HCC animal model, mice are treated for 2 weeks with either (A) Copaxone (B) IL-2 (C) Copaxone and IL-2 and (D) no additional treatment. These treatment protocols are followed in mice that underwent hepatic fibrosis induced mice, resulting in four additional groups (E), (F), (G) and (H), respectively. Each group includes 10 male animals.

Mice are followed for survival and tumor size for 2 weeks following splenocytes reconstitution. Following Ketamine/Xylazine anesthesia, animals were sacrificed, and serum, livers and cells are harvested 3 days after the final dose of CCl₄. Blood samples are obtained and frozen at −20° C. until assayed for HBsAg, anti-HBs (see below), and AFP levels. Splenocyte subpopulations are analyzed by FACS for CD4 and CD8, NK markers, and killer inhibitory and activation receptors. Livers are assessed for fibrosis severity.

(IV) The Immune Therapy of Inflammatory Bowel Disease:

Following colitis induction, mice are treated for 12 days with either (A) Copaxone (B) IL-2 (C) Copaxone and IL-2 and (D) no additional treatment. Each group includes 10 male animals.

Following Ketamine/Xylazine anesthesia, animals were sacrificed, and serum, colons are harvested at day 12. Blood samples are obtained and frozen at −20° C. until assayed for cytokine levels (IL2, IL4, IFNg, IL10 and TGFb). Splenocyte subpopulations are analyzed by FACS for CD4 and CD8, NK markers, and killer inhibitory and activation receptors. Colons are assessed for inflammatory severity.

Animal Models:

Hepatic fibrosis animal model: Hepatic fibrosis was induced by IP CCl₄ (10% as diluted with corn oil) administration 5 microliter/g body weight; for 4-6 weeks in 8 week-old male mice.

HCC animal model: Hepatitis B virus (HBV)-associated HCC expresses HBsAg on its cell surface and serves as a tumor-associated antigen. Recipients Balb/c mice (Harlan USA) are kept in laminar flow hoods in sterilized cages, and receive irradiated food and sterile acidified water. The mice are conditioned with sub-lethal radiation (600 cGy). At 24 hours after irradiation, animals are subcutaneously injected in the right shoulder with 107 human hepatoma Hep3B cells (expressing HBsAg). Seven days after irradiation, athymic mice receive mixture of 80% bone marrow cells and 20% spleen cells at 2×10⁶ cells/mouse [Ilan, Y., et al. J. Hepatology, 27:170-176, 1997] and are then followed for further 2 weeks.

Colitis induction as inflammatory bowel disease animal model: TNBS-colitis was induced by rectal instillation of TNBS, 1 mg/mouse, dissolved in 100 μl of 50% ethanol as described [Trop S, Samsonov D, Gotsman I, Alper R, Diment J and Ilan Y (1999) Hepatology 29: 746-755].

Methods

Liver and colon histology: The posterior one-third of the liver, the rectosigmoid colon were fixed in 10% formalin for 24 hours and then paraffin-embedded in an automated tissue processor. Seven-millimeter sections were cut from each animal specimen. Hematoxylin and eosin (H&E) staining was performed for each animal section.

Liver sections (15 μm) were stained in 0.1% Sirius Red F3B in saturated picric acid (both from Sigma). Additionally, alpha smooth muscle actin immunohistochemistry was performed using the DAKO kit (CAT# U7033 EPOS, Monoclonal) according to the manufacturer's instructions.

Hepatic Fibrosis quantitation: Relative fibrosis area (expressed as a % of total liver area) was assessed by analyzing 36 Sirius red-stained liver sections per animal. Each field was acquired at 10× magnification and then analyzed using a computerized Bioquant® morphometry system. To evaluate the relative fibrosis area, the measured collagen area was divided by the net field area and then multiplied by 100. Subtraction of vascular lumenal area from the total field area yielded the final calculation of the net fibrosis area.

Splenocyte isolation: Spleens were harvested at the time of sacrifice and fractionated through a 70-μm nylon cell strainer. After RBC lysis, splenocytes were washed, suspended in RPMI 1640 medium and stored at 4° C. until FACS analysis.

Fluorescent-activated cell sorting analysis (FACS): splenocytes are analysed by direct immunofluorescence reactivity with a series of antibodies (Abs) using standard techniques on a Coulter flow cytometer (BECTON DICKINSON, USA). Briefly, 3×10⁵ spleen cells are incubated for 30 minutes at 4° C. with Abs conjugated to fluorescein isothiocyanate (FITC), phycoerythrin (PE) or allophycocyanin (APC), washed three times, and resuspended in fixative solution with 2% paraformaldehyde for analysis. Antibodies used for staining splenocytes are monoclonal anti-mouse CD4, CD8 conjugated by PE and FITC respectively (BD Biosciences). For staining natural killer (NK) cells APC-conjugated rat anti-mouse CD49b/Pan-NK cells monoclonal antibody which identifies the majority of NK cells is used. For killer inhibitory receptors (iKIR) monoclonal mouse anti-mouse Ly-49C and Ly-49I conjugated by PE are used. For the killer activating receptor (aKIR) FITC-conjugated rat anti-mouse LY-49D monoclonal antibody were used. To defined the role of T regulatory cells we used the anti CD25 monoclonal antibodies. To identify lymphocytes staining with peridinin chlorophyll-α protein (Per-CP)-conjugated rat anti-mouse CD45 (BD Biosciences) was used. Lymphocyte subsets are presented as percentage of the CD45+ cells.

Radioimmunoassays for detection of HBsAg, anti-HBs and alpha-feto-protein: HBsAg and antibodies to HBsAg are determined by a commercial solid phase radio immunoassay (RIA) (Ausria II and Ausab, Abbott Laboratories, North Chicago, Ill.). A World Health Organization reference serum is used for quantitative analysis of anti-HBs by RIA, utilizing the Hollinger formula and data expressed in mIu/ml (22). Alpha feto protein (AFP) is measured by RIA (AFP, Bridge Serono, Italy) and expressed in ng/ml.

Alpha smooth muscle actin immunoblot: Immunoblot analysis of Alpha-SMA in liver extracts is performed as previously described [de Waal Malefyt, R., et al., J. Exp. Med. 1991; 174:915-924] with modifications. Whole-liver protein extracts were prepared in liver homogenization buffer (50 mmol/L Tris-HCl [pH 7.6], 0.25% Triton-X 100, 0.15 M NaCl, 10 mM CaCl₂ and complete mini EDTA-free protease inhibitor cocktail (Roche Diagnostics, Mannheim, Germany). Then proteins (30 μg per lane) were resolved on a 10% SDS-polyacrylamide gel under reducing conditions. For immunoblotting, proteins were transferred to a Protran membrane and incubated overnight at 4° C. in a blocking buffer containing 5% skim milk. Anti-SMA mouse monoclonal antibody (DAKO, cat# M0851) and peroxidase-conjugated goat anti-mouse IgG (P.A.R.I.S., Compiègne, France) and enhanced chemiluminescence were used.

Statistics: Student's test was used for statistically significant differences.

Results:

(I) The Role of NK Cells in Hepatic Fibrosis:

Relative fibrosis area was: CCl₄ WT 2.32% (±1.39) of total liver area, CCl₄ SCID 1.11% (±6.4), CCl₄ SCID BIEGE 1.85% (±1.19), Naive WT 0.3% (±0.37). Hepatic fibrosis was significantly increased in all CCl₄ treated groups compared to controls (P=0.001). SCID-BEIGE group had a significant increase of fibrosis compared to the SCID Group (P=0.0001). Relative fibrosis correlated closely with α-SMA expression in all groups. Following fibrosis in WT mice, splenocyte FACS analysis revealed significant decreases of CD4 cells from 26%±5.7 to 19%±3.8 (P=0.01), and a significant increase of the αKIR:iKIR ratio in NK cells from 0.76±0.21 to 2.79±0.98 (P=0.003) without significant alterations in absolute NK, CD11c cells and class I presentation. Similar results were obtained in the analysis of the intrahepatic lymphocytes (IHL). These findings indicate a functional activation of NK cells following fibrosis induction. Concomitantly, the Class-I molecules average expression in hepatic stellate cells (HSC) significantly decreased from 81%±15 to 37%±12, P=0.001 (a decrease to 51%±13 of basal values) following fibrosis. HSC Class-II+ molecules expression did not vary. The decrease of the self-recognition marker of class-I on activated HSC reflects an enhanced susceptibility to killing by activated NK cells.

(II) The Immune Therapy of Hepatic Fibrosis:

The effect of the immune therapy was evaluated by the measuring liver injury and fibrosis in the CCl4 treated animals, using the following parameters:

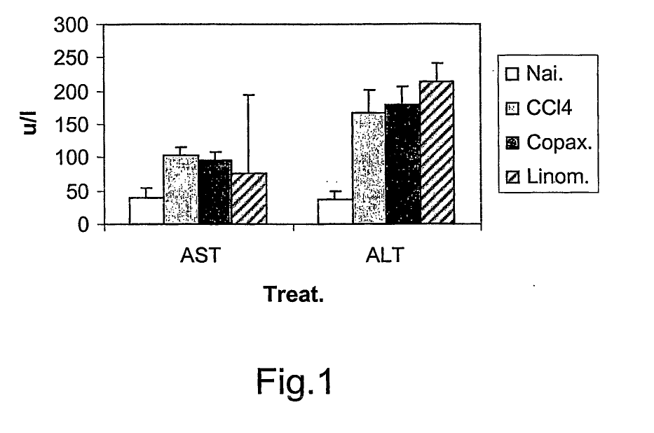

Liver injury severity Liver injury estimated by AST and ALT serum levels, was higher in all the experimental groups treated with CCl4 as compared to naïve mice. No significant different could be seen among the different groups in which fibrosis was induced (FIG. 1).

Evaluation of different liver histopathology parameters representative of the inflammatory and necrotic condition of the liver tissue by the Ishak injury score, was found not to be different among the groups (FIG. 2).

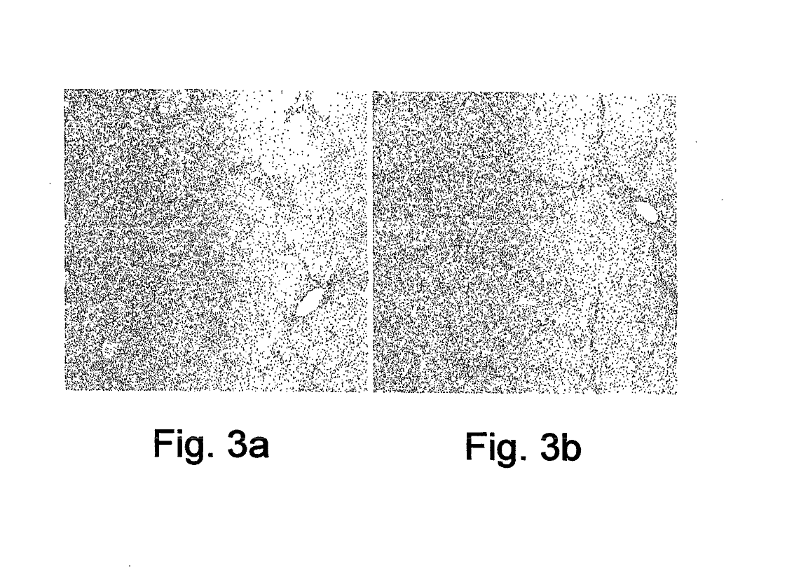

Fibrosis severity score Fibrosis severity was evaluated by Bioquant analysis of hepatic H&E sections collected from the different groups (see example in FIG. 3) and a smooth muscle actin (α-SMA) quantitation.

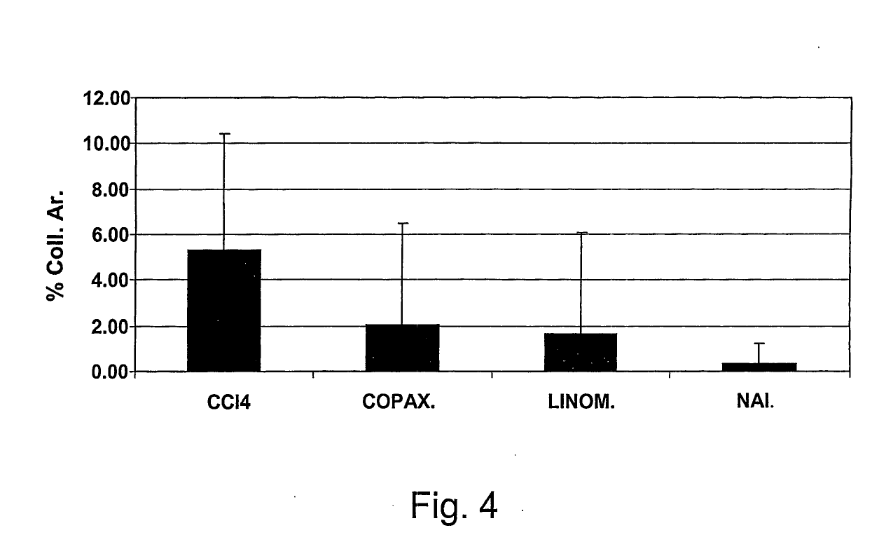

Fibrosis severity estimated by the % of collagen in the analyzed area was significantly reduced (as seen in FIG. 4) in the groups treated with Copaxone or Linolin after CCl4 fibrosis induction (p<0.0001).

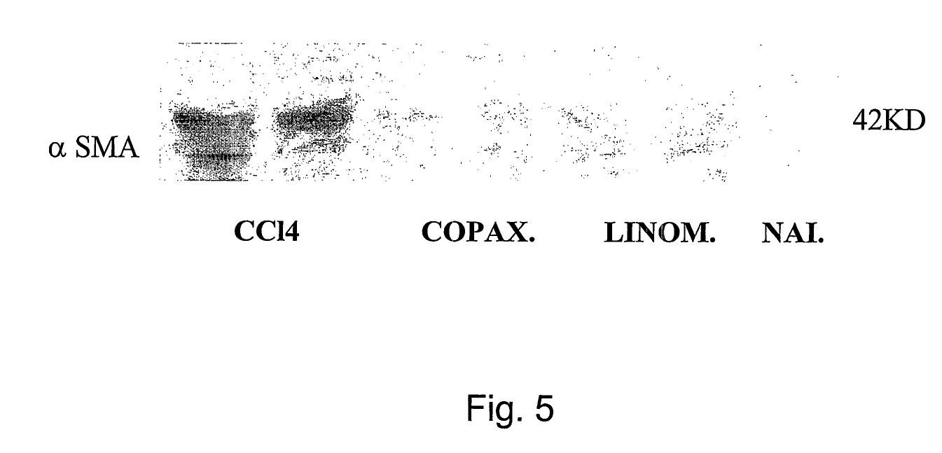

Alpha smooth muscle actin amounts correlated with the fibrosis extend, being highly expressed in the CCl4 animal group and significantly reduced in the Copaxone and Linolin groups (FIG. 5).

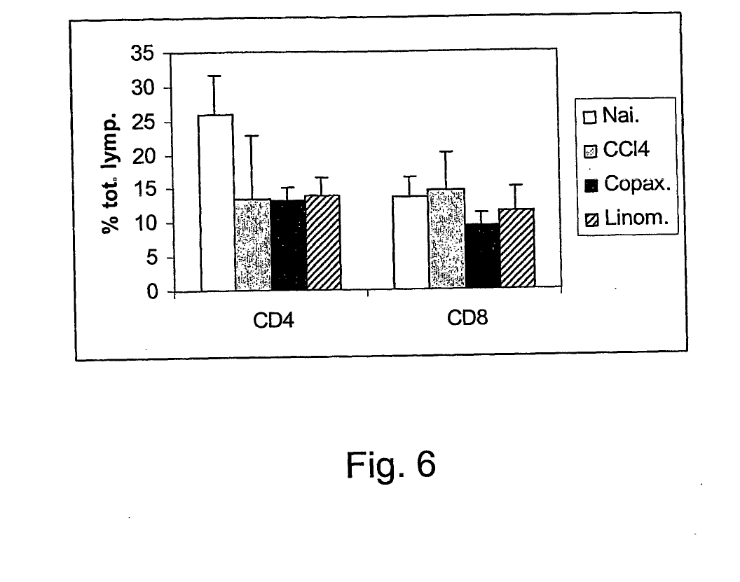

Lymphocyte cell populations FACS analysis of splenocyte revealed significant decrease of CD4 cells (p<0.0005) following fibrosis induction in all fibrotic groups (A, B and C), but no significant changes were seen in between (FIG. 6).

CD8 cells significantly decreased in group A compared to either C (p<0.05) or D (p<0.005) groups (FIG. 6). CD4:CD8 ratio significantly decreased following fibrosis in the C group (p<0.0003) (FIG. 7). The ratio was significantly elevated in the two treated groups (A and B), but still significantly lower that the naïve animals (group D) (p<0.001 and p<0.016 respectively). CD4/CD8 ratio in the group A was significantly higher than in group C (p<0.04) (FIG. 7).

The total number of NK cells did not change following fibrosis (groups C and D), however, both treated groups (A and B) showed an increased number that was significant among the copaxone group (p<0.002) (FIG. 8). aKIR:iKIR ratios were calculated while aKIAR and iKIR were measured as total reading and as specific readings on the NK cells. Although both showed same pattern, the latter was more specific as the ratio significantly increased following fibrosis in all groups (A, B and C) as seen in FIG. 9. A and B groups showed significant increase of the NK aKIR:NK iKIR ratio (p<0.0001) compared to the naïve group (group D), but still significantly lower compared to the untreated fibrotic group (group C) (p<0.05).

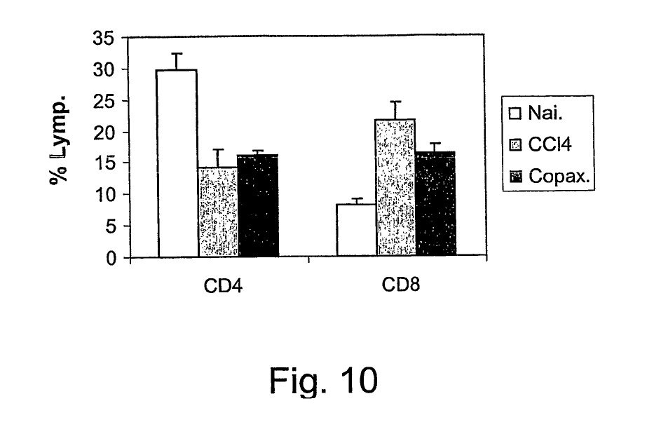

FACS analysis of intrahepatic lymphocytes revealed a significant decrease (p<0.000001) of CD4 cells following fibrosis induction in the fibrotic groups (A and C), but no significant changes were seen in between (FIG. 10).

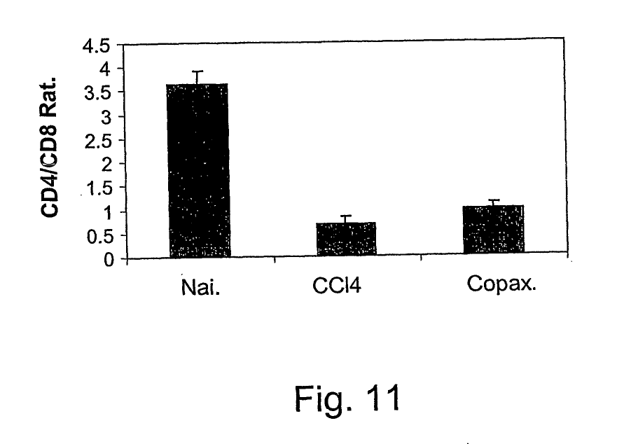

CD8 cells significantly decreased in group A compared to C (p<0.001) but it was significantly higher than group D (p<4×10⁻⁷) (FIG. 10). CD4:CD8 ratio significantly decreased following fibrosis in the C group (p<3.1×10⁻¹⁰) (FIG. 11). The ratio was significantly elevated in the treated group A compared to group C (p<0.002) but still significantly lower to that ratio in the naïve animals (group D) (p<4.2×10⁻¹⁰) (FIG. 11).

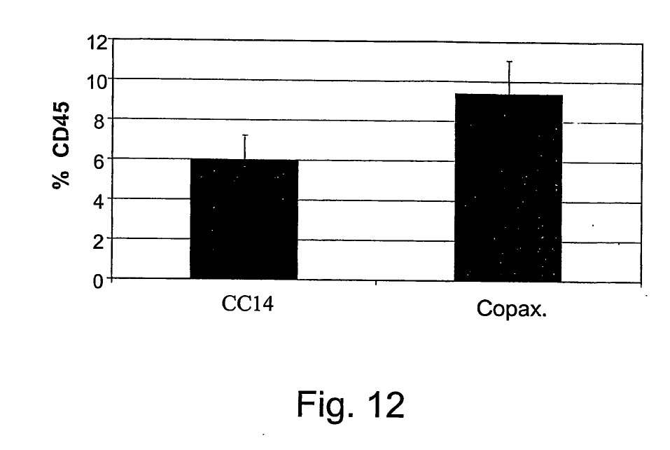

The intrahepatic NK cells number was significantly increased among the copaxone group (p<0.0001) as compared to the fibrosos group C (FIG. 12).

Conclusions: The above experiments show that Copaxone and Linomycin had a significant anti-fibrotic effect in the used animal model. This effect was mediated by increasing the CD4:CD8 ratio that increased the total number of NK cells. Those NK cells were activated and stimulated against the activated stellate cells and thus decreased fibrosis. This conclusion is strongly supported by the histopathological findings which showed no significant difference in the liver injury of the copaxon group (A) and the CCl4 group (C) as measured using the Ishak score method. Therefore, copaxone reduces fibrosis not by directly reducing the inflammatory process, but rather by instigating a decrease on HSC activation.

(IV) The Immune Therapy of Inflammatory Bowel Disease

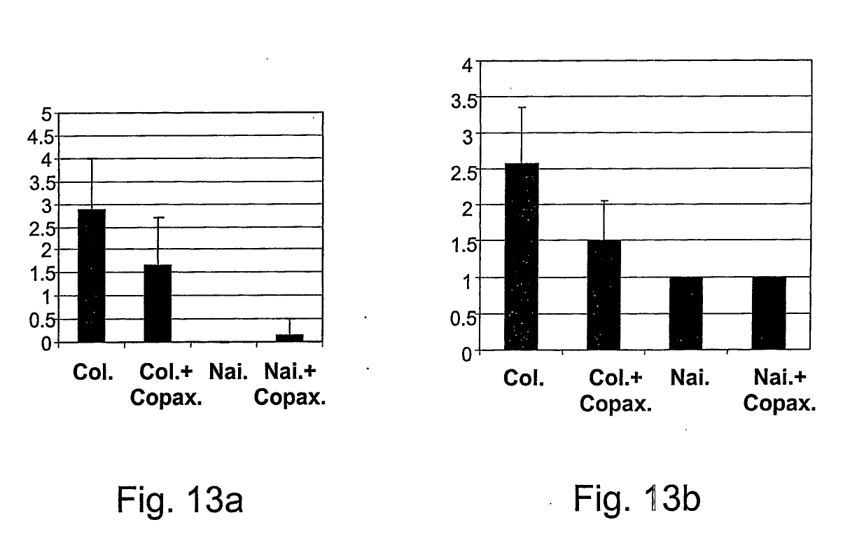

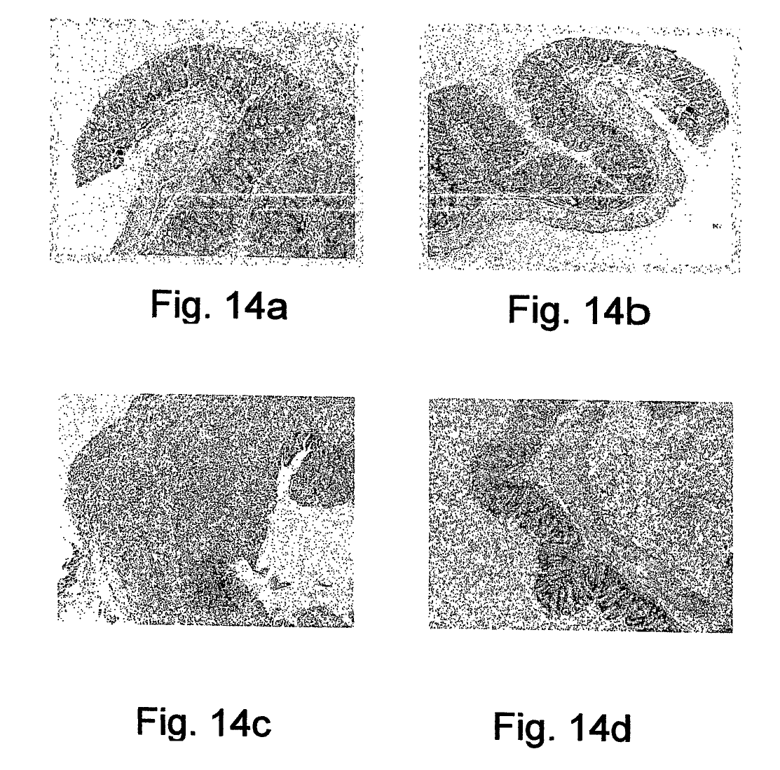

Following colitis induction mice were treated with Copaxone. Macroscopic and microscopic colitis scores were compared between the treated and control groups. In both examinations, there was a prominent reduction in tissue injury as a consequence of the Copaxone treatment (FIGS. 13a and 13b). Microscopic improvement is especially evident as exemplified in FIG. 14 (p<0.03).

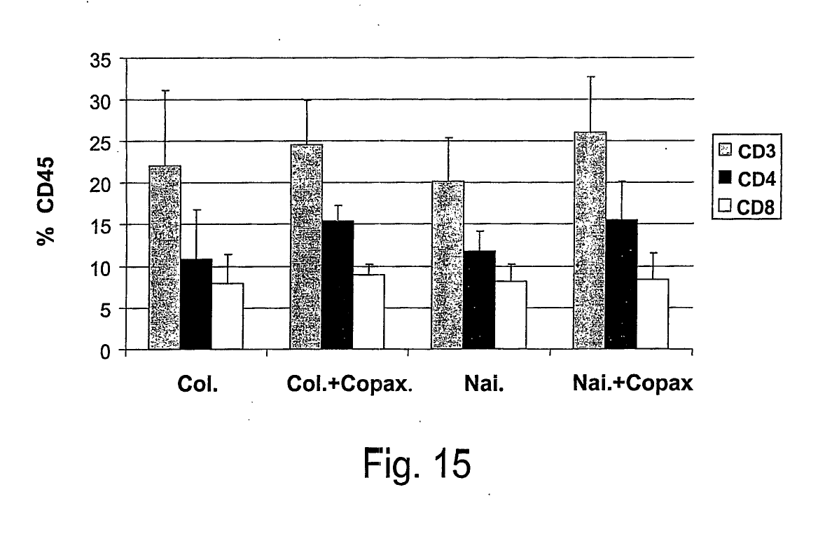

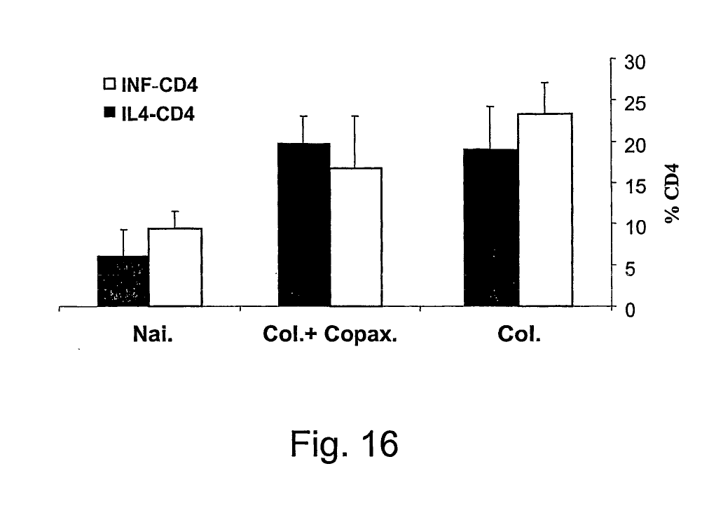

FACS analysis of the spleen CD3, CD4 and CD8 cell populations showed a significant increase (p<0.03) of the CD4 cell number in the colitis induced-Copaxone treated group (FIG. 15). The enlarged CD4 cell number was accompanied by an increase of the Interferon-γ (IFN-γ) (p<0.02), but not of the Interleukin-4 (IL-4), secreting cells (FIG. 16).

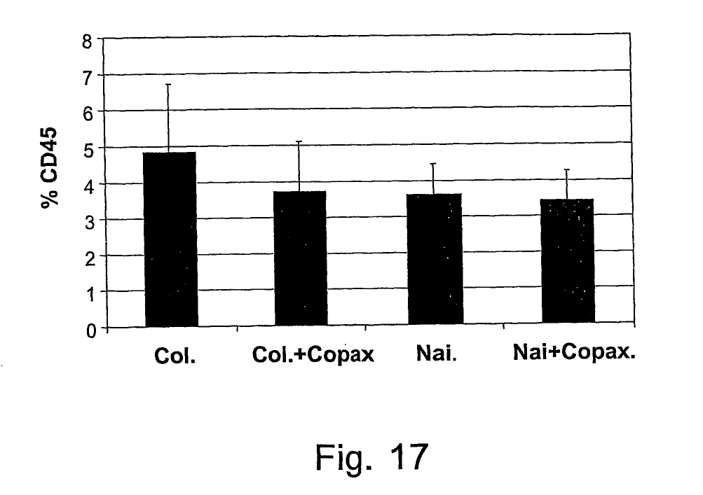

CD8 cell populations were unaffected either by the colitis induction neither by the Copaxone treatment (FIG. 15). Antigen presenting cells (APC) seemed to be reduced after Copaxone treatment (FIG. 17).

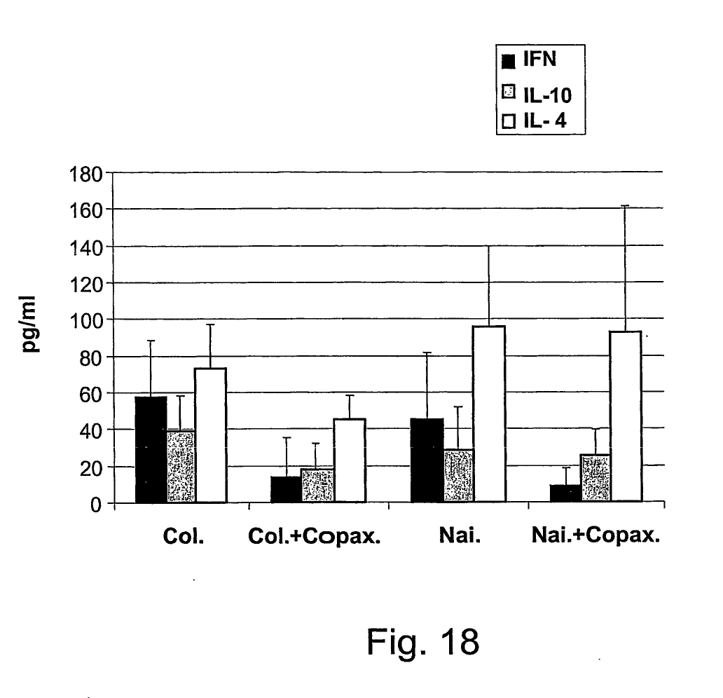

The measured serum cytokine IFN-γ, IL-4 and IL-10 levels, were significantly reduced in the colitis induced-Copaxone treated group (p<0.006, p<0.004 and p<0.002 respectively) when compared to the colitis-induced untreated group (FIG. 18).

Conclusions: Copaxone treatment reduces the tissue injury of the colitis affected animals by changing their immuno-modulatory status. Copaxone administration influenced the T cell CD4 population structure and the Th1 and Th2 cytokine profiles.

1. A method for the treatment of hepatic fibrosis comprising administering to a subject in need thereof a therapeutically effective amount of an immuno-modulatory agent.

2. The method of claim 1, wherein said immuno-modulatory agent elevates the CD4:CD8 ratio and/or increases the number of NK cells and/or elevates the NK aKIR:NK iKIR ratio in hepatic tissue.

3. The method of claim 1, wherein said immuno-modulatory agent is glatiramer acetate.

4. The method of claim 1, wherein said subject is a human subject.

5.-7. (canceled)

8. An immuno-modulatory agent for use in the treatment of hepatic fibrosis.

9. The immuno-modulatory agent of claim 8, wherein said agent elevates the CD4:CD8 ratio and/or increases the number of NK cells and/or elevates the NK aKIR:NK iKIR ratio in hepatic tissue.

10. The immuno-modulatory agent of claim 8, being glatiramer acetate.

11. A method of elevating CD4:CD8 ratio and/or increasing the number of NK cells and/or elevating NK aKIR:NK iKIR ratio in hepatic tissue comprising administering to a subject in need of such treatment an immuno-modulatory agent that elevates the CD4:CD8 ratio and/or increases the number of NK cells and/or elevates the NK aKIR:NK iKIR ratio in hepatic tissue.

12. The method of claim 11, wherein said immuno-modulatory agent is glatiramer acetate.

13. The method of claims 11 for the treatment of hepatic fibrosis.

14. (canceled)

15. A method for screening for an immuno-modulatory agent which is useful in the treatment of hepatic fibrosis, comprising the steps of:
a. providing a test agent;
b. providing a fibrosis-induced model animal and a non-fibrotic model animal;
c. administering said test agent to said fibrotic and non-fibrotic animals;
d. obtaining samples of hepatic tissue from said animals;
e. measuring at least one of the following parameters: CD4:CD8 ratio, NK cell number, NK aKIR:NK iKIR ratio, area of the fibrotic tissue in said sample and at least one accepted fibrosis parameter; and
f. comparing the results obtained for the sample obtained from the fibrosis-induced animal with the corresponding results obtained for the non-fibrotic animal;
whereby elevation of CD4:CD8 ratio, and/or increased number of NK cells, and/or elevated NK aKIR:NK iKIR ratio, and/or reduced area of the fibrotic tissue and/or decrease of said accepted fibrosis parameter indicates that said test agent is useful in the treatment of hepatic fibrosis.

16. The method of claim 15, wherein said accepted fibrosis parameter is Ishak fibrosis score, computerized Bioquant® quantitation or alfa smooth muscle actin assessment using Western blotting.

17. A method for the treatment of hepatic cellular carcinoma comprising administering to a subject in need thereof a therapeutically effective amount of at least one immuno-modulatory agent.

18. The method of claim 17, wherein said method comprises at least one immuno-modulatory agent that elevates the CD4:CD8 ratio and/or increases the number of NK cells and/or elevates the NK aKIR:NK iKIR ratio in hepatic or bowel tissue.

19. The method of claim 17, wherein said immuno-modulatory agent is glatiramer acetate, optionally in combination with IL-2.

20. The method of claim 19, wherein said subject is a human subject.

21. A method for the treatment of inflammatory bowel disease comprising administering to a subject in need thereof a therapeutically effective amount of at least one immuno-modulatory agent.

22. The method of claim 21, wherein said method comprises at least one immuno-modulatory agent that elevates the CD4 cell number and/or reduces the number of IFN-γ CD4 secreting cells but not IL-4 CD4 secreting cells and/or reduces the Il-4, IL-10 and IFN-γ concentration in serum or bowel tissue.

23. The method of claim 21, wherein said immuno-modulatory agent is glatiramer acetate, optionally in combination with IL-2.

24. The method of claim 21, wherein said subject is a human subject.

25. A method for screening for an immuno-modulatory agent which is useful in the treatment of inflammatory bowel disease, comprising the steps of:
a. providing a test agent;
b. providing a colitis-induced model animal and a non-colitis model animal;
c. administering said test agent to said colitis and non-colitis animals;
d. obtaining samples of bowel tissue from said animals;
e. measuring at least one of the following parameters: the CD4 cell number, the IFN-γ CD4 and IL-4 CD4 secreting cells number, Il-4, IL-10 and IFN-γ concentration in serum or bowel tissue, histopathological exam of the inflammed tissue in said sample; and
f. comparing the results obtained for the sample obtained from the colitis-induced animal with the corresponding results obtained for the non-colitis animal;
whereby elevation of the total CD4 cell number and/or the reduction of the of the IFN-γ CD4 but not IL-4 CD4 secreting cell number and/or reduction of the Il-4, IL-10 and IFN-γ concentration in serum or bowel tissue and/or improvement of the histopathological condition indicates that said test agent is useful in the treatment of inflammatory bowel diseases.

26.-29. (canceled)

30. An immuno-modulatory agent for use in the treatment of any one of hepatic cellular carcinoma and inflammatory bowel disease.

31. The immuno-modulatory agent of claim 30, wherein said agent elevates the CD4:CD8 ratio and/or increases the number of NK cells and/or elevates NK aKIR:NK iKIR ratio in hepatic tissue.

32. The immuno-modulatory agent of claim 30, wherein said agent elevates the CD4 cell number and/or reduces the number of IFN-γ CD4 secreting cells but not IL-4 CD4 secreting cells and/or reduces the Il-4, IL-10 and IFN-γ concentration in serum or bowel tissue.

33. The immuno-modulatory agent of claim 30, being glatiramer acetate, optionally in combination with IL-2.
